(19) United States
(12) Patent Application Publication
(10) Pub. No.: US 2016/0089316 A1
(43) Pub. Date: Mar. 31, 2016

(54) Compound, Composition, Polymer, Mylar Film and Method for Indicating Maximum Exposure to the Sun and UV Radiation

(71) Applicant: Genesis Laboratories Inc., Wilmington, DE (US)

(72) Inventor: Clifton Sanders, Carrollton, TX (US)

(73) Assignee: GENESIS LABORATORIES INC., Wilmington, DE (US)

(21) Appl. No.: 14/742,209

(22) Filed: Jun. 17, 2015

Related U.S. Application Data
(60) Provisional application No. 62/055,946, filed on Sep. 26, 2014.

(51) Int. Cl. A61K 8/49; A61K 8/40; A61K 8/31; A61Q 17/04

(52) U.S. Cl. CPC A61K 8/498; A61Q 17/04; A61K 8/40; A61K 8/31; A61K 2800/45; A61K 2800/56; A61K 2800/592

(57) ABSTRACT

A composition for protecting from ultraviolet radiation is disclosed. The composition comprises a filter and an effectiveness indicator responsive to the reduction of effectiveness protection.

### RELATED PRIORITY DATE APPLICATION

This application claims the benefit under 35 U.S.C. 119(e) of the U.S. provisional application No. 62/055,946 filed on Sep. 26, 2014.

### TECHNICAL FIELD OF THE INVENTION

The present invention relates to the field of protecting the skin from the harmful effects of UVA and UVB radiation and, more particularly, to sunscreen compositions. Still more particularly, the present invention discloses sunscreen compositions that include indicators that indicate the effectiveness of the sunscreen with the passage of time after its application to the skin.

### BACKGROUND OF THE INVENTION

The skin care industry has long recognized that sunlight exposure increases the risk of wrinkles, age spots, skin sagging and directly causes sunburn and is a known cause of skin cancer. Such skin damage is photochemical in nature and is associated with high energy, short wavelength radiation. This leads to undesirable biochemical changes, such as inflammation and DNA and cell organelle damage.

The skin care industries position regarding ultra violet radiation has evolved to include protection against both ultra violet A and ultra violet B radiation. It has been known for some time that UVB, while enabling the skin to produce Vitamin D3, nevertheless also produces erythema (sunburn). If the UVB radiation reaches a threshold dose level termed the minimal erythemal dose (MED), sufficient UVB radiation has been delivered to the skin to cause visible erythema. UVA radiation is orders of magnitude less erythmogenic than UVB radiation, but is nevertheless damaging to the skin. The basis of this position was evidence of DNA damage caused by UVA radiation that penetrates deeper into the skin. Therefore, with regard to ultra violet radiation damage to the skin form the sun, the prevailing view is that both UVB and UVA radiation should be blocked to prevent damage to the skin.

UVB sunscreens are evaluated by their ability to prevent erythema, which is how the Sun Protection Factor (SPF) is typically defined. Because UVA radiation does not redden the skin (erythema), its damaging effects cannot be determined by current SPF testing. However, UVA light because of its longer wavelength can penetrate deeper into the skin than UVB light and is considered a prime cause of long term damage such as wrinkles, collagen damage which causes skin sagging and DNA damage. It is therefore very important that both UVA and UVB radiation be blocked from the skin.

Present sunscreen formulations typically include a mixture of compounds for absorbing UVA and UVB radiation. Commercially approved formulations include a UVB blocker, such as p-methoxycinnamate, or an amino benzoate and a UVA blocker, such as benzene or an anthranilate. These compounds generally absorb an incoming UV photon and reradiate a lower energy photon. Once all of the available photons have been used, the sunscreen is no longer effective.

A need therefore exists in the art to provide compounds and compositions, polymers and mylar films that indicate when a sunscreen no longer has available absorbable photons to protect the skin and needs to be reapplied. Accordingly, provided herein are new compounds and formulations that have an aesthetically pleasant clear application and turn blue when the limit for photon absorption has been reached according to the SPF factor on the indicator itself, or accompanying sunscreen. The same can be said for an applied polymer or mylar film with the present formulation attached to its surface, and all embodiments can be used under or over the sunscreen. These and other advantages of the present invention will become apparent from the following description.

### SUMMARY OF THE INVENTION

The purpose of this invention is the creation of a cosmetic cream/lotion/gel, hydro alcoholic solu-tion and anhydrous compound in addition to polymer and mylar films that will indicate the presence of ultra-violet light when the invention is applied onto human skin. It does so by changing color(s) when exposed to ultra violet light in the range between 280 to 400 nanometers. In so doing it will indicate the amount of ultra violet exposure to human skin.

The invention is a mixture of photochromic inks and liquid crystals that have either been encapsulated in shells with particle sizes between 5 to 1500 microns or as free flowing inks and crystals.

The capsule is characterized by a clear non-pigmented shell surrounded by a single droplet or particle of a hydrophobic core material either with or without color. Photochromism is the reversible transformation of chemical species between two forms, by the absorption of electromagnetic radiation where the two forms have different absorption spectra.

A photochromic complex is a chemical compound that has a photo responsive part on its ligand. These complexes have a specific structure: photo switchable organic compounds are attached to metal complexes. For the photo controllable part, thermally and photochemically stable chromo pores (azobenzene, diarylethelene, spiropyran) are used.

### DETAILED DESCRIPTION OF THE PREFERRED EMBODIMENTS

A reduction in the harmful effects of sunlight and ultra violet radiation, e.g. sunburn and skin cancer, occupies greater consumer emphasis than ever before. In June of 2014, The Surgeon General of the United States issued a call to action to prevent the rising problem of skin cancer. The incorporation of UVA and UVB filters in cosmetic and OTC (over the counter) products of various types is long standing. For example, skin care and cosmetic products containing known filters such as, octocrylene, avobenzone, oxybenzone, homosalate, padimate O, titanium dioxide and zinc oxide, among others are available commercially. However, it is impossible to know when it is time to re-apply such products. In other words, after quantitative exposure to sunlight and UV radiation, when are they no longer effective and when exactly should the consumer re-apply to avoid sunburn and potential skin cancer.

The present invention therefore is directed to providing compounds and compositions in addition to polymer and mylar films that can be applied to the skin with adhesives that overcome the problems associated with overexposure to UVA and UVB radiation.

The present invention can be applied prior and beneath a commercially available sunscreen of any SPF value to indicate when it is no longer active or protecting and allowing dangerous levels of UV radiation through to the skin, above the sunscreen or independently as a sun exposure indicator. The present invention can be incorporated into a sunscreen composition or as an indicator alone in forms such as creams, gels, sprays, sticks, polymer and mylar films.

Specifically, compounds, compositions, polymer and mylar films of the present invention reduce the risk of skin damage from the harmful effects of sunlight and sunburn by containing encapsulated ink and liquid crystals that change color(s) from clear to blue after the designated amount of UV radiation has been absorbed. The present compounds also reduce the risk of prolonged exposure to the sun and reduce the risk of premature aging of the skin, such as wrinkles and other visual indicia of skin aging, like the loss of skin tones and elasticity and sun spots as well as long term DNA damage.

It is clear that the ability to indicate when a sunscreen is no longer active or capable of absorbing UV radiation is a vast improvement to current methods and compositions.

In many embodiments, a compound to protect the skin from UVA and UVB radiation can be added to the composition. Because both UVA and UVB radiation can be damaging, a preferred sunscreen provides protection from both types of radiation. In such embodiments, the compositions protect all layers of the skin UVB and/or UVA filters typically are used in sunscreen compositions of the present invention. sunscreens can be organic and inorganic compounds.

The photochromic inks and liquid crystals are added to an organic ester and surfactants to ensure their compatibility to the following ultra violet absorbers and blockers: p-Aminobenzonic acid, padimate O, phenylbenzimidazole sulfuric acid, cinoxate, dioxybenzone, homosalate, octocrylene, octyl methoxycinnamate, octyl salicylate, sulisobenzone, trolamine salicylate, avobenzone, ecamsule, 4-methylbenzyladine camphor, tinosorb M, tinosorb S, neo heliopan AP, mexoryl XL, benzophenone-9, uninul T 150, uninul A plus, uvasorb HEB, parasol SLX, amiloxate, oxybenzone (benzophenone-3), tannic acid, uric acids, quinine salts, dihydroxy naphthalin acid, and PEG-25 PABA. Further, sunscreen compounds such as dioxybenzone, cinoxate, ethyl-4[bis(hy-droxypropyl)]amino benzoate, glycerol amino benzoate, homosalate, menthol anthranilate, octocrylene, octyl methoxycinnamate, octyl salicylate, padimate A, padimate O, red petrolatum, titanium dioxide, 4-menthylbenzylidene camphor, benzophenone-1, benzophenone-2, benzophenone-4, benzophenone-6, benzophenone-12, isopropyl dibenzoylmethane, butyl methoxydi-benzoylmathane, zotocrylene, DEA-methoxycinnamate, digalloyl trioleate, TEA-salicylate, or zinc oxide can be used in the current composition.

Therefore, variations on the present compositions include a UV-specific compound to absorb or block UVA and/or UVB radiation to protect the skin. These compounds filter a percentage of the UV spectrum depending on the type, concentration, and intensity of the chromophores used.

The filter families include, alone or in combination, and not limited to, the benzotriazoles, benzophenomes, benzoic acids/PABA, cinnamates, salicylates, and avobenzones, to further protect the skin against UVS and UVB damage. Maximum loads of one or more UV filters in a sunscreen composition can be up to 15-30% by weight. A UVA and UVB filter, individually, are present in an amount of about 0.25 to about 3 percent by weight of the composition. when both a UVA and UVB blocker are used, each typically is present in an amount of about 0.5 to about 1.5 percent, by weight.

Additional UV filters, including combinations of any two or more, are selected from the following categories (with specific examples): p-aminobenzoic acid, its salts and its derivatives (ethyl, isobutyl, glyceryl esters; p-dimethylaminobenzoic acid); anthranilates (o-aminobenzoates; methyl, menthol, phenyl, benzyl, phenyl ethyl, linalyn, terpinyl, and cyclohexenyl esters); salicylates) octyl, amyl, phenyl, benzyl, menthyl, (homosalate) glyceryl, and dipropyleneglycol esters); cinnamic acid derivatives (menthyl and benzyl esters; alpha-phenyl cinnamonitrate; butyl cinnamoylpyruvate); dihydroxycinnamic acid derivatives (umbelliferone, methylumbelliferone, methylaceto-umbelliferone); camphor derivatives (3 benzylidene, 4 methylbenzylidene, polyacrylamidomenthyl benzylidene, benzalkonium methosulfate, benzylidene camphor sulfuric acid, and terephthalyidene dicamphor sulfuric acid); trihydroxycinnamic acid derivatives (esculetin, methylesculetin, daphnetin, and glucosides, esculin and daphnin); hydrocarbons (diphenylbutadiene, stilbene); dibenzalacetone; benzalacetophenone; naphtholsulfonates (sodium salts of 2-naphthol-3,6-disulfonic and of 2-naphthol-6,8-disulfonic acids); dihydroxy-naphthoic acid and its salts; o- and p-hydroxydiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric acid derivatives; vilouric acid derivatives, tannic acid and its derivatives; hydroquin; and benzophenones (oxybenzone, sulisobenzoone, dioxybenzone, benzoresorcinol, octabenzone, 4-isopropyldibenzoylmethane, butylmethoxydibenzoylmethane, etocrylene, and 4-isopropyl-dibenzoylmethane).

The following are additional specific UV filters: 2 ethylhexyl p-methoxycinnamate, 4,4-t-butyl methoxydibenzoylmethane, octyldimethyl p-aminobenzoate, digalloyltrioleate, ethyl 4-[bis(hydroxypropyl)]aminobenzoate, 2-ethylhexylsalisylate, glycerol p-aminobenzoate, 3,3,5-trimethylcyclohexylsalisylate, and combinations thereof.

Sunscreen or dermatological formulations can include a variety of photoactive compounds, preferably including one or more UVA photoactive compound and one or more UVB photoactive compound. In many embodiments, a sunscreen or dermatological formulation includes a photoactive compound selected from the group consisting of p-aminobenzoic acid and sals and derivatives thereof; anthranilate and derivatives thereof; dibenzoylmethane and derivatives thereof; salicylate and derivatives thereof; cinnamic acid and derivatives thereof; dihydroxycinnamic acid and derivatives thereof; camphor and salts and derivatives thereof; trihydroxycinnamic acid and derivatives thereof; dibenzalacetone naphthosulfonate and salts and derivatives thereof; benzalacetophenone naphtholsulfate and salts and derivatives thereof; dihydroxy-naphthoic acid and salts and derivatives thereof; o-hydroxydiphenyldisulfonate and salts and derivatives thereof; p-hydroxydiphenyldisulfonate and salts and derivatives thereof; coumarin and derivatives thereof; diazole derivatives; quinine derivatives and salts thereof; quinoline derivatives, uric acid derivatives; vilouric acid derivatives; tannic acid and derivatives thereof; hydroquinone; diethyl amino hydroxybenzoyl hexyl benzoate and salts and derivatives thereof; and mixtures thereof.

The above described UV filters are commercially available. For example, suitable commercially available organic UV filters are identified by trade name and supplier in Table 1 below.

In addition to UVA and UVB filters, a present composition can also contain a photo stabilizer for the UVA and UVB filters. Photostabilizers include octocrylene, methylbenzilydene camphor, and esters and polyesters of naphthalene dicarboxolic acid. Alkoxy crylenes, and notably methoxy crylenes, are useful photo stabilizers.

An important feature of the present invention is a topically applied compound for providing a cosmetic or therapeutic effect, in addition to a compound for protection from radiation, can be any of a wide variety of compounds either water soluble or oil soluble, as well as polymer or mylar films.

More specifically, such compounds can be one of or a mixture of, a cosmetic compound, a medicinally active compound, a compound used in cosmetics or personal care, or any other compound that is useful upon topical application to the skin. Such topically applied agents include, but are not limited to, skin care compounds, plant extracts, antioxidants, insect repellents, counterirritants, vitamins, steroids, antibacterial compounds, anti fungal compounds, anti-inflammatory compounds, topical anesthetics, sunscreens, and other cosmetic and medicinal topically effective compounds and the above mentioned polymers and mylar films.

For example, a skin conditioner could be the topically applied compound skin conditioning agents include, but are not limited to, humectants, such as a fructose, glucose, glycerin, propylene glycol, glycereth-26, mannitol, pyrrolidone carbolic acid, hydrolyzed lecithin, coco-betaine, cysteine hydrochloride, glucamine, sodium gluconate, potassium aspartate, oleyl betaine, thiamine hydrochloride, sodium laureth sulfate, sodium hyaluronate, hydrolyzed proteins, hydrolyzed keratin, amino acids, amine oxides, water-soluble derivatives of vitamin A, E and D, selenium and derivatives thereof, amino-functional silicones, ethoxylated glycerin, alpha-hydroxy acids and salts thereof, fatty oil derivatives, such as PEG-24 hydrogenated lanolin, beta-hydroxy acids and salts thereof) e.g., glycol acid, and salicylic acid), and mixtures thereof. Numerous other skin conditioners are listed in the CTFA cosmetic ingredient handbook, First Ed., J. Nikotakis, ed., The Cosmetic, Toiletry and Fragrance Association (1988), (hereafter, CTFA Handbook) pages 79-84, incorporated herein by reference.

The skin conditioner can also be a water-insoluble ester having at least 10 carbon atoms, and preferably 10 to about 32 carbon atoms. Suitable esters include those comprising an aliphatic alcohol having about 8 to about 20 carbon atoms and an aliphatic or aromatic carboxylic acid including from 2 to about 12 carbon atoms, or conversely, an aliphatic alcohol having about 2 to about 12 carbon atoms with an aliphatic or aromatic carboxylic acid including about 8 to about 20 carbon atoms. The ester is either straight-chained or branched. suitable esters therefore include, for example, but are not limited to:

(a) aliphatic monohydric alcohol esters, including, but are not limited to: myristyl propionate, isopropyl isostearate, isopropyl myristate, isopropyl palminate, acetyl acetate, cetyl-propionate, cetyl-stearate, isodecyl neopentatonoate, cetyl-octonoate, isocetyl stearate;
(b) aliphatic di- and tri-esters of polycarboxylic acid, including but not limited to: diisopropyl adipate, diisostearyl fumarate, dactyl adipate, a succinate ester, and triisostearyl citrate;
(c) aliphatic polyhydric alcohol esters, including but not limited to: propylene glycol dipelargonate;
(d) aliphatic esters of aromatic acids, including but not limited to: C12-C15 alcohol esters of benzoic acid, octylsalicylate, sucrose benzoate, and dactyl phthalate. Numerous other esters are listed in the CFTA Handbook, at pages 24 through 26, incorporated herring by reference.

The topically applied compound can also be retinoid acid or a retinol derivative and can be applied to polymer or mylar films.

Similarly, topically applied drugs, like anti fungal compounds, antibacterial compounds, anti-inflammatory compounds, topical anesthetics, skin rash, skin disease and dermatitis medications, and anti-itch and irritation reducing-reducing compounds can be used as an active agent of the present composition of the present invention. For example, analgesics such as benzocaine, dyclonine hydrochloride, also vera and the like; anesthetics such as butambem picrate, lidocaine hydrochloride, xylocaie and the like: antibacterials and antiseptics such as povidone-iodine, polymyxin b sulfate-bacitracin, zinc-neomycin sulfate-hydrocortisone, chloramphenicol, ethylbenzethonium chloride, erythromycin and the like; anti parasitics such as lindane and essentially all dermatologicals like acne preparations such as benzoyl peroxide, erythromycin benzoyl peroxide, clindamycin phosphate, 5,7-dichloro 8-hydroxyquinoline and the like; burn relief ointments such as o-amino-p-toluensulfonamide mono acetate and the like; dipigmenting agents such as monobenzone; dermatitis relief agents such as the active steroid amcinomide, diflorasone dictate, hydrocortisone and the like; emollients and moisturizers such as mineral oil, PEG-4 dilaurate, lanolin oil, petrolatum, mineral wax and the like; fungicides such as butocouazole nitrate, haloprogin, clotrimazole and the like; herpes treatment drugs such as O-[(2-hydroxymethyl)-methyl]guanine; pruritic medications such as alclometasone dipropionate, betanethasone vakerate, isopropyl myristrate MSD and the like; psoriasis, seborrhea and scabicide agents such as anthralin methoxsalen, coal tar and the like; steroids such as 2-(acetyloxy)-9flouro-1′,2′,3′,4′-tetrahydro-11-hydroxypregna-1,4-dieno-[16,17-b]naphthalene-3,20-dione. Any other medication capable of topical administration like skin protectants such as allontoin and anti-acne agents such as salicylic acid can leo be incorporated in the composition of the present invention in the amount sufficient to perform its intended function. Other topically applied compounds are listed Remington's Pharmaceutical Sciences, 17th Ed., Mack Publishing Co., Easton, Pa. (1985), pages 773-791 and pages 1054-1058 (hereafter Remington's), incorporated herein by reference.

The topically active compound can also be a plant extract or natural oil. Preferred plant extracts and natural oils absorb blue light and generally have a yellow, yellow-brown, or red color. Numerous plant extracts are available form Lipo Chemicals, In. Paterson, N.J. Nonlimiting plant extracts are those obtained from alfalfa, aloe vera, alma fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickweed, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon licorice root, lime malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root. An example of a natural oil is rice bran oil.

The present composition can also contain a surfactant. The surfactant can be an anionic surfactant, a cationic surfactant, a nonionic surfactant or a compatible mixture of surfactants. The surfactant can also be an ampholytic or amphoteric surfactant which have anionic or cationic properties depending upon the pH of the composition.

Examples of anionic surfactants include, without limitation, soaps, alkyl sulfates, anionic acyl sarcosinates, methyl acyl taurates, N-acyl glutamates, acyl isethionates, alkyl phosphate esters, ethoxylated alkyl phosphate esters, alkyl sulfosuccinates, trideceth sulfates, protein sondensates, mixtures of ethoxylated alkyl sulfates and the like. Examples of anionic non soap surfactants include, without limitation, the alkali metal salts of an organic sulfate having an alkyl radical containing about 8 to about 22 carbon atoms and a sulfuric acid or sulfuric acid ester radical. Examples of zwitterionic surfactants include, without limitation, derivatives of aliphatic quaternary ammonium, phosphonium, and sulfonium compounds in which the aliphatic radicals can be straight chain or branched and wherein one of the aliphatic substituents contains an anionic water-solubilizing group, e.g., carboxyl, sulfonate, sulfate, phosphate or phosphonate. Examples of amphoteric surfactants include, without limitation, derivatives of aliphatic secondary and tertiary amines in which the aliphatic radical can be straight chained or branched and wherein one of the aliphatic substituents contains about 8 to about 18 carob atoms and one contains an anionic water solubilizing group, e.g., carboxyl, sulfonate, sulfate, phosphate or phosphonate. Examples of cationic surfactants include without limitation, stearyldimethylbenzyl ammonium chloride; dedecyltrimethyl ammonium chloride; nonylbenzylethyldimethyl ammonium nitrate and tetradecylpyridium bromide. Nonionic surfactants include without limitation, compounds produced by the condensation or ethylene oxide groups with an organic hydrophobic compound, which may be aliphatic or alkyl aromatic in nature, for example, the polyethylene oxide condensates of alkyl phenols.

A present composition can also contain hydrotrope. A hydrotrope is a compound that has an ability to enhance the water solubility of other compounds. specific examples of hydrotropes include but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid and xylene sulfonic acid. Other useful hydrotopes include sodium polynaphthalene sulfonate, sodium polystyrene sulfonate, sodium methyl naphthalene sulfonate, sodium camphor sulfonate, and disodium succinate.

A present composition can also contain an organic solvent, for example, as a component of the cosmetically acceptable barrier. The solvent can be a water-soluble organic compound containing one to six, and typically one to three hydroxyl groups, e.g., alcohols, diols, triols, and polyols. Specific examples of solvents include, but are not limited to, methanol, ethanol, isopropyl alcohol, n-butanol, n-propyl alcohol, propylene glycol, glycerol, diethylene glycol, dipropylene glycol, tripropylene glycol, hexylene glycol, butylene glycol, 1,2,6-hexanetriol, sorbitol, PEG-4,1,5-pentanediol, similar hydroxyl-containing compounds and mixtures thereof. The solvent can also be water or an aprotic solvent, e.g., dimethyl sulfide or tetrahydrofuran.

A present composition can also contain a thickening or gelling agent. A thickening or gelling agent can be, for example, a polymer that is water soluble or that generates a colloidal solution water. A thickening or gelling agent, therefore, can be, for example, polymers or copolymers unsaturated carboxylic acids or unsaturated esters, polysaccharide derivatives, gums, colloidal salicates, polyethylene glycols (PEG) and their derivatives, polyvinylpyrrolidones and their derivatives, polyacrylamides and their derivatives, polyacrylonitriles, hydrophilic silica gels or mixtures thereof.

Specific thickening or gelling agents can be, for example, acrylic and/or methacrylic polymers or copolymers, vinyl carboxylic polymers, polyglyceryl acrylates or methacrylates, polyacrylamides derivatives, cellulose or starch derivatives, chitin derivatives, alginates, amino acids, ceramides, fatty acids, cholesterol and derivatives thereof and other natural moisturizing compounds, hyaluronic acid and its salts, chondroitin sulphates, xantham, gellan, Rhamsan, karaya or guar gum, carob flour and colloidal aluminum magnesium silicates of the montmorillonite type.

Additional thickening or gelling agents include vinylcarboxylic polymers sold under the trade name CARBOPOL (Lubrizol/Noveon), acrylic acid/ethyl acrylate copolymers, acrylic acid/stearyl methacrylate copolymers, carboxymethylcellulose, hydroxymethylcellulose, hydroxypropylcellulose, microcrystalline cellulose hydroxypropyl guar, colloidal rectorates, bentonites and the like.

The present composition can also contain pigments, dyes, preservatives, hydrating agents and the like.

The pigments can be inorganic pigments, organic pigments, or nacreous pigments. Inorganic pigments include but are not limited to, titanium dioxide, black, yellow, red or brown oxide, manganese violet, ultramarine violet, ultramarine blue, chlorium oxide, and the like. Among organic pigments, non limiting examples include D& C Red No. 3, No. 6, No. 7, No. 9, No. 13, No. 19, No. 21, No. 27, No. 30, or No. 38, or alternatively carbon black.

The nacreous pigments can be, for example, white nacreous pigments such as mica coated with titanium oxide or with bismuth oxychloride. Colored nacreous pigments, such as titanium mica colored with iron oxides or with chromium oxide, titanium mica colored with an organic pigment of the above mentioned type, or alternatively, nacreous pigments based on bismuth oxychloride can also be used.

The dye can be, for example, a water-soluble dye, such as ponceau disodium salt, alizarin green disodium salt, quinoline yellow amaranth trisodium salt, tartazine disodium salt, rhodamine monosodium salt, fuchsin do sodium salt, xanthophylls and the like.

The present composition can contain fillers, such as clays of the montmorillonite, rectorate or bentonite type, or other fillers such as silicas, silicone powders, polyamides or powdered polymathy methacrylate. Various white fillers such as, for example, talc, kaolin, powdered TEFLON (polytetraflouroethylene), powdered polyethylene, powdered cross linked poly0beta-alanine and the like.

Other classes of optional ingredients included in a present composition can be, but not limited to, pH adjusters, chelating agents, preservatives, buffering agents, foam stabilizers, opacifiers and similar classes of ingredients known to persons skilled in the art. Specific optional ingredients include inorganic phosphates, sulfates, and carbonates as buffering agents; EDTA and phosphates as chelating agents; and acids and bases as pH adjusters.

Nonlimiting examples of basic pH adjusters are ammonia; mono-di-, and tri-alkyl amines; mono-, di-, and tri-alkylanolamines; alkali metal and alkaline earth metal hydroxides; and mixtures thereof. Specific, non limiting examples of basic pH adjusters are ammonia, sodium, potassium and lithium hydroxide; monoethanolamine; triethylamine; isopropanolamine; diethanolamine and tiethanolamine. Examples of acidic pH adjusters are mineral acids and organic carboxylic acids. Nonlimiting examples of mineral acids are citric acid, hydrochloric acid, nitric acid, phosphoric acid and sulfuric acid.

A composition of the present invention is topically applied to the skin as needed in gel, cream, lotion, polymer or mylar films to indicate when a sunscreen of given SPF value needs to be reapplied and thereby protect the skin from the harmful effects of UV radiation. Typically, the composition is topically applied to the skin every 10 minutes to 90 minutes depending if the skin has no SPF sunscreen applied or the maximum protection currently commercially sold. However, application of the present composition can be more or less frequent as prescribed, required or desired. The present compositions are applied to the skin by spraying, or rubbing. The preferred route of administration is rubbing onto the skin with a soft massage to ensure ultimate contact with the skin.

To demonstrate the new and unexpected benefits provided by the present invention, numerous compositions were prepared with components in the ranges indicated below. The appearance of each composition was noted, and the absorbance spectra and time elapsed for the composition to change color from clear to blue were taken and analyzed. Each examples group represents several examples of compositions in the ranges indicated therein. The examples further illustrate the invention but are not to be construed as limitations on the scope of the invention contemplated herein.

### Examples Group 1

### Anhydrous Indicator Formulations without Sunscreen

| | |
|---|---|
| Phochromic Microcapsule Slurry | 0.1% to 10% |
| Microencapsulated Photochromic Dye, | 0.1% to 10% |
| Aqueous Dispersion | |
| Liquid Crystal | 0.1% to 10% |
| Natural or Synthetic Waxes | 0.1% to 30% |
| Natural or Synthetic Oils | 0.1% to 30% |
| Aliphatic Esters (Ethoxylated) | 0.1% to 30% |

### Examples Group 2

### Anhydrous Indicator Formulations with Sunscreen

| | |
|---|---|
| Phochromic Microcapsule Slurry | 0.1% to 10% |
| Microencapsulated Photochromic Dye, | 0.1% to 10% |
| Aqueous Dispersion | |
| Liquid Crystal | 0.1% to 10% |
| Natural or Synthetic Waxes | 0.1% to 30% |
| Natural or Synthetic Oils | 0.1% to 30% |
| Aliphatic Esters (Ethoxylated) | 0.1% to 30% |
| Sunscreen | 0.1% to 30% |
| Silicon Fluids | 0.1% to 30% |

### Examples Group 3

### Lotion Indicator Formulations with Sunscreen

| | |
|---|---|
| Phochromic Microcapsule Slurry | 0.1% to 10% |
| Microencapsulated Photochromic Dye, | 0.1% to 10% |
| Aqueous Dispersion | |
| Ethoxylated Oil | 0.1% to 10% |
| Ethoxylated Silicon Fluids and Clays | 0.1% to 30% |
| Water | 5% to 30% |
| Glycols/Humectants | 1% to 5% |
| Liquid Crystal | 1% to 5% |
| Sunscreen | 1% to 30% |

### Examples Group 4

### Gel Indicator Formulations with Sunscreen

| | |
|---|---|
| Phochromic Microcapsule Slurry | 0.1% to 10% |
| Microencapsulated Photochromic Dye, | 0.1% to 10% |
| Aqueous Dispersion | |
| Ethoxylated Oil | 1% to 10% |
| Water | 5% to 30% |
| Glycols/Humectants | 1% to 5% |
| Liquid Crystal | 1% to 5% |
| Neutralizer | 0.1% to 2% |
| Gelling Agent | 0.5% to 2% |
| Seppi Gel (Acrylate Polymer) | .5% to 2.5% |
| Sunscreen | 1.0% to 30% |

While the invention is described with respect to specific embodiments, modifications thereof can be made by one skilled in the art without departing from the spirit of the invention. The details of said embodiments are not to be construed as limitations except to the extent indicated in the following claims.

What is claimed is:

1. A composition for protecting from ultraviolet radiation comprising a filter and an effectiveness indicator responsive to the reduction of effectiveness protection.

2. A composition according to claim 1 wherein the effectiveness indicator is a photochromic dye.

3. A composition according to claim 1 wherein the composition is a gel.

4. A composition according to claim 1 wherein the composition is a lotion.

5. A sunscreen comprising a sunscreen filter and an effectiveness indicator.

6. A sunscreen according to claim 5 wherein the effectiveness indicator is a photochromic dye.

7. A sunscreen according to claim 6 wherein the photochromic dye is microencapsulated.

8. A composition comprising a phochromic microcapsule slurry and a microencapsulated photochromic dye.
